(19) United States
(12) Patent Application Publication
(10) Pub. No.: US 2022/0111322 A1
(43) Pub. Date: Apr. 14, 2022

(54) Passenger Compartment Air Vent Filter

(71) Applicant: Ramsis Benjamin, Coeur d'Alene, ID (US)

(72) Inventor: Ramsis Benjamin, Coeur d'Alene, ID (US)

(21) Appl. No.: 17/492,800

(22) Filed: Oct. 4, 2021

Related U.S. Application Data
(60) Provisional application No. 63/091,542, filed on Oct. 14, 2020.

(51) Int. Cl. B01D 46/00; B01D 46/42; A61L 9/20; B64D 13/06

(52) U.S. Cl. CPC B01D 46/0028; B01D 46/0038; B01D 46/0002; B01D 46/4254; B01D 46/4245; B64D 2203/00; B64D 13/06; B01D 2279/50; B01D 2279/35; A61L 2209/12; B64D 2013/0651; A61L 9/20

(57) ABSTRACT

The present invention relates generally to a passenger compartment air vent filtering device. More specifically, the invention includes a cover that can be placed over top of an existing air vent in a passenger seating compartment to filter the air flowing therefrom, and comprises both a HEPA filter and a UV light disinfection filter. In a preferred embodiment, the novel air vent filtering device further comprises a moveable element, a plurality of LED lights and a scent diffuser.

### CROSS-REFERENCE TO RELATED APPLICATION

The present application claims priority to and the benefit of U.S. Provisional Application No. 63/091,542 filed on Oct. 14, 2020, which is incorporated herein by reference in its entirety.

### FIELD OF THE INVENTION

The present invention relates generally to an air flow filtering device for a seating area in a passenger compartment. More specifically, the invention relates to an air filtering cover that can be placed over top of an airplane cabin seat vent. The air filtering cover is secured to the air vent such that all of the air being expelled through the vent passes through the body of the air filtering cover. The air filtering cover is preferably comprised of both a replaceable HEPA filter and a UV light disinfection filter. Further, the air exits the cover via a permeable element that comprises a fixed or removable cap on one end of the cover. The cap may include a plurality of patterns, designs, textures, or physical structures, such as flower petals, bristles or the like. Further, the cap may be scent-infused to permeate the cabin air with a scent after the vent air passes through the air filtering cover. Accordingly, the present disclosure makes specific reference thereto. However, it is to be appreciated that aspects of the present invention are also equally amenable to other like applications, devices, and methods of manufacture.

### BACKGROUND OF THE INVENTION

In the age of a global pandemic, health experts warn individuals against gathering in enclosed spaces as they may be more easily exposed to the spread of germs, bacteria, pathogens and viruses, such as COVID-19 or any other virus that can be transmitted through the air. However, some enclosed spaces, such as on airplanes, buses, trains and the like, cannot be avoided by individuals who are required to travel for work or other purposes. Further, in an airplane, bus, train, etc., the passenger or cabin air is typically recycled throughout the cabin after passing through one or more cabin filters. Unfortunately, cabin filters do not always remove all of the bacteria, pathogens, viruses or germs that may be circulating in the cabin air and passenger space. Further, many individuals choose to utilize the air conditioning and heating vents that are disposed overhead in the passenger convenience portion of a seat. Air vent openings may be found in the overhead passenger convenience area, alongside walls such as in buses, trains or other vehicles, on a seat back or wherever a transportation vehicle manufacturer may position the vent. As such, passengers may come into direct contact with pathogens and other airborne contaminants in the recycled cabin air via the vent as it blows air into their personal area. No device currently exists that allows a user to easily apply a cover and filter devices over a passenger cabin air vent.

Therefore, there exists a long felt need in the art for a cabin seat air vent cover device that protects an individual from being directly exposed to airborne pathogens and other undesirable contaminants in a passenger cabin that may be expelled through the passenger's personal air vent. There is also a long felt need for an air vent cover device having redundancy filtering mechanisms to further reduce the possibility that an individual will be exposed to germs, bacteria, pathogens and viruses. Moreover, there is a long felt need in the art for an air vent cover device that is effective, yet relatively small and portable for easy transportation. Additionally, there is a long felt need in the art for an air vent cover device that not only filters the air, but that also infuses the filtered air with a pleasant scent. Finally, there exists a long felt need in the art for an air vent cover device that is relatively inexpensive to manufacture, and that is both safe and easy to use.

The present invention relates to a relatively small and generally cylindrical air filtering device that can be placed over an air vent in a passenger cabin. The air filtering device creates a seal around the exterior portion or body of the vent to ensure that all air passing through the vent enters into and travels through the various filters of the air filtering device. More specifically, the device is comprised of a replaceable HEPA filter and a UV light disinfection filter that both help to significantly reduce, and potentially eliminate, any germs, bacteria, pathogens and viruses that may be present within the air that passes through the device. Additionally, the top of the air filtering device is comprised of a cap having a permeable filtering element that, in differing embodiments of the device, may include elements such as artificial flowers, bristles, characters, geometric shapes, etc. The permeable filtering element may further be infused with a scent that is carried out of the device via the air passing therethrough, thereby eliminating odors from stale cabin air. Differing embodiments of the device may include a fixed or removable top cap wherein the device may also be provided as part of a kit that contains a plurality of caps having differing decorative elements, replacement filters, scent packages, instructions and the like.

In this manner, the passenger compartment air vent filter device of the present invention accomplishes all of the forgoing objectives and allows a user to better protect himself or herself from airborne pathogens, germs, viruses, microbes and other contaminants in a passenger cabin, such as an airplane cabin, that may otherwise be expelled through an air vent and directly onto the user. Further, the device is small and portable, and can easily be carried in a pocket of a user. Finally, the passenger compartment air vent filter device is comprised of a top that can include a decorative element which makes the device aesthetically pleasing, and allows the user to customize the appearance of the device to suit his or her needs or preferences.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one embodiment thereof, comprises an improved passenger compartment air vent-cover filtering device. The filtering device is comprised of a shaped, flexible body portion that can be placed around, and secured to, the adjustment ring or opening of a passenger cabin air vent to form a seal therewith that ensures that all of the air being expelled through the vent will travel though the filtering device body. The filtering device body has an internal passageway which includes a plurality of continuous slots or openings that can house one or more air filters, such as a replaceable HEPA filter and a UV light filter. The slots or other openings are preferably arranged in a fashion directly above one another (e.g. stacked) such that the top surfaces of each of the filters are generally parallel to one another and have air passageways which are generally aligned with the internal air passageway of the filter body. In one embodiment of the filtering device, each of the filters may be secured within the body via a plurality of tabs upon which the filters rest. In another embodiment, the body may comprise one or more pull-out trays that, when in the open position, extend outwardly from the filter device body such that each filter can be placed within its individual tray and the tray pushed back into the body of the device. Each slot or opening is further covered via a hinged cover that is preferably made from the same material as the device body, and generally convex in shape in order to sit flush with the outer surface of the device body.

After being placed on and secured around the air vent on the transportation vehicle, the expelled air will pass through the internal passageway of the filtering device of the present invention and through each of the filters to purify the air and eliminate any bacteria, germs, contaminants or pathogens that may be circulating within the passenger compartment or cabin air. The filtering device is further comprised of a decorative or aesthetically pleasing cap that in differing embodiments may be any number of permeable decorative elements such as, but not limited to, flower petals/flowers, bristles, geometric shapes, airplanes, buses, train cars, a flat surface that has a plurality of patterns, textures, designs or combinations thereof. Further, the decorative cap may be fixedly attached to the device body or removable therefrom, thereby allowing the cap to be cleaned or for the user to interchange differing caps with differing design elements.

In a further embodiment of the present invention, each decorative element or the HEPA filer itself may be infused with a scent so that the air passing through the filter and the element retains the specific scent as it is discharged into the passenger cabin, thereby eliminating the smell of stale cabin air. The end cap may also comprise an ornamental component that moves as the air flows through the same, such as a small pinwheel, mobiles or the like, which can be used to provide entertainment to a child or infant while strapped in a passenger seat. The end cap may also be equipped with one or more LED lights, which can be used for supplemental reading light.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

FIG. 1 illustrates a perspective view of one potential embodiment of a passenger compartment air vent filtering device of the present invention in accordance with the disclosed architecture and having a decorative cap;

FIG. 2 illustrates a perspective view of one potential embodiment of a decorative cap for a passenger compartment air vent filtering device of the present invention in accordance with the disclosed architecture;

FIG. 3 illustrates a perspective view of one potential embodiment of a passenger compartment air vent filtering device of the present invention in accordance with the disclosed architecture, wherein the two filter trays are in the open position so that a filter may be loaded therein;

FIG. 4 illustrates a perspective view of one potential embodiment of a passenger compartment air vent filtering device of the present invention in accordance with the disclosed architecture, wherein the filtering device is about to be installed on an air vent in a passenger cabin of a public transportation vehicle;

FIG. 5 illustrates a perspective view of one potential embodiment of a passenger compartment air vent filtering device of the present invention in accordance with the disclosed architecture, wherein the filtering device is installed on an air vent in a passenger cabin of a public transportation vehicle;

FIG. 6 illustrates a perspective view of one potential embodiment of a passenger compartment air vent filtering device kit of the present invention in accordance with the disclosed architecture; and

FIG. 7 illustrates a perspective view of one potential embodiment of a passenger compartment air vent filtering device of the present invention in accordance with the disclosed architecture, wherein the cap further comprises an accessory that moves as air is passing therethrough.

### DETAILED DESCRIPTION OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

As noted above, there is a long felt need in the art for a passenger cabin air vent cover device that protects an individual from being directly exposed to airborne pathogens and other undesirable contaminants, and that has redundancy filtering mechanisms to further reduce the possibility that an individual will be exposed. Moreover, there is a long felt need in the art for an air vent cover device that is relatively small and portable, and that not only filters the air but also infuses the filtered air with a pleasant scent. Finally, there exists a long felt need in the art for an air vent cover device that may also be used to entertain a small child on long trips or commutes, and that is relatively inexpensive to manufacture.

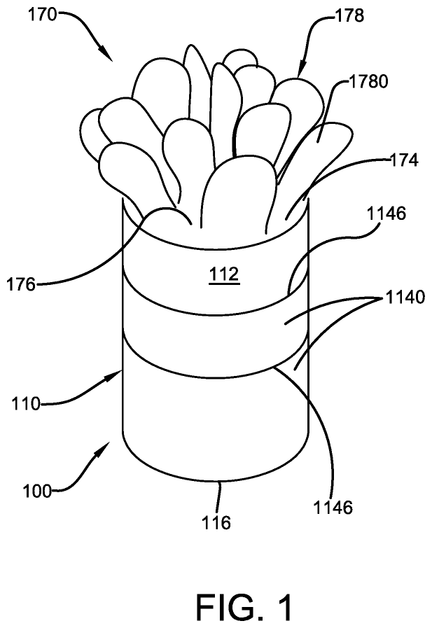

Referring initially to the drawings, FIG. 1 illustrates a perspective view of one potential embodiment of a passenger compartment air vent filtering device 100 of the present invention in accordance with the disclosed architecture and having a decorative cap 170. More specifically, in a preferred embodiment, the air vent filtering device 100 comprises a body portion 110, a HEPA filter 130, a UV filter 150, a battery 160 and a decorative cap 170. The body portion 110 is a generally cylindrical, relatively small and hollow structure that is preferably made from a soft rubber material. As explained more fully below, the body portion 110 houses the HEPA filter 130 and the UV filter 150, each of which is removable, replaceable and interchangeable, as well as the battery 160. Notwithstanding, it is also contemplated that the body portion 110 may be rigid, semi-rigid, or flexible, and may further be comprised of an anti-bacterial exterior coating, such as triclosan, to further prevent the transmission of disease. It is also contemplated that the body portion 110 may be molded from a plastic or other suitable material with the antibacterial agent (not shown) fully embedded in the material as part of the manufacturing process.

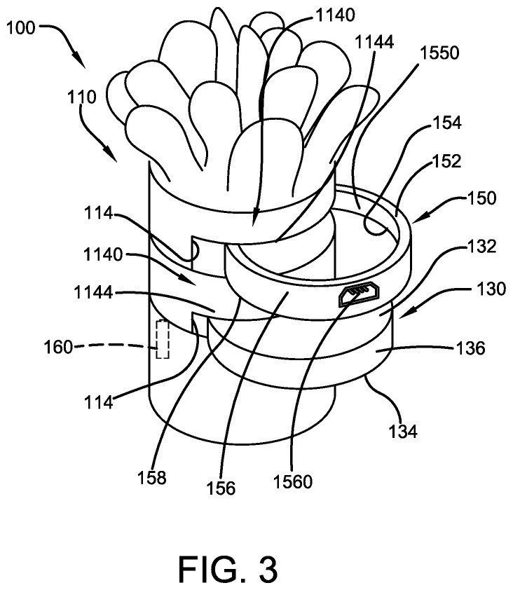

The body portion 110 is further comprised of a relatively flat and generally circular bottom surface 116 with a continuous opening therein for engaging an air vent 10 opening as described more fully below. The body portion 110 is also comprised of a plurality of continuous opening filter slots 114 which can accommodate a plurality of filters such as first and second filters 130, 150, as described more fully in relation to FIG. 3. Once the filters 130, 150 have been inserted into the slots 114, the same are enclosed with a cover 1140 that is preferably made from the same material, and generally in line with, the body portion 110, as best shown in FIG. 1. Each cover 1140 is preferably fixedly attached to the outer surface 112 of the body 110 via a hinge 1146 that comprises the top edge of the cover 1140. In a preferred embodiment, the hinge 1146 is made from the same material as the body portion 110, and is preferably integrally formed with the body portion 110 during the manufacturing process (e.g. via injection molding or additive manufacturing). Each of the covers 1140 have a curved convex front surface 1142 that rests on or sits flush with the outer surface 112 of the body portion's generally cylindrical shape when in a closed position, as best shown in FIG. 1.

As noted above, the air vent filtering device 100 also comprises a decorative end cap 170 that fits over the top of the body portion 110, as best shown in FIGS. 1, 3, 4 and 5. In differing embodiments of the air filtering device 100, the cap 170 may be fixedly or removably attached to the body portion 110. For example, in the fixedly attached embodiment, the cap 170 is comprised of a side surface 174 that is relatively flush or inline with the outer surface 112 of the body portion 110. In contrast, in a removable cap embodiment, the side surface 174 of the cap 170 may be larger in diameter than the diameter of the body portion 110, such that the body portion 110 fits partially within the cap 170 and the side surface 174 covers a portion of the body portion 110. In the removable embodiment, the cap 170 is secured to the body 110 via a friction or interference fit between the portion of the side surface 174 covering the outer surface 112 of the body portion. Nonetheless, in either embodiment, the connection of the decorative cap 170 to the body portion 110 must be strong enough to withstand the air pressure created by the air passing through the air vent 10 and the air filtering device 100 so that the cap 170 doesn't get blown off of the end of the body portion 110.

Additionally, the cap 170 is comprised of a top surface 176 that includes a permeable decorative element 178. In one embodiment of the device 100, the element 178 may be a plurality of colorful artificial flower petal-like protrusions 1780 that give the device 100 an overall aesthetically pleasing appearance. Nonetheless, it is contemplated that the decorative element 178 may vary in differing embodiments of the device 100, and a user may further be able to swap out or exchange caps 170 that are constructed from differing decorative elements 178 based on the wants and needs of the user.

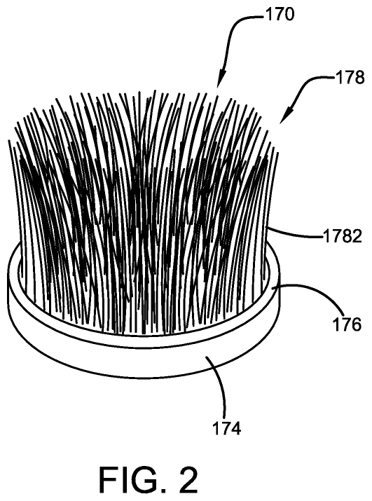

For example, FIG. 2 illustrates a perspective view of one potential embodiment of a decorative cap 170 for a passenger compartment air vent filtering device 100 of the present invention in accordance with the disclosed architecture and that differs from the design of the decorative element 178 shown in FIG. 1. More specifically, in this embodiment of the cap 170, the decorative element 178 is comprised of a plurality of bristles 1782 that form the top surface 176 of the cap 170. It is further contemplated that in any embodiment of the device 100, the decorative element 178 may further include a scented compound that releases a scent when air from the vent 10 permeates through the element 178, and thereby releases the scent into the passenger compartment or cabin. The scent may be imbedded into the decorative element 178, or may be provided through one or more of the removable filter elements disposes in the openings or slots, or may be added by a scent dispenser (not shown). The scent may be any one of a number of different scents such as, but not limited to, lavender, peppermint, spearmint, citrus, eucalyptus, clove, tea tree, evergreen, sandalwood, vanilla, cedarwood or a combination thereof.

Further, the decorative element 178 may take on a plurality of known appearances in differing embodiments of the device 100 such as, but not limited to, flowers/flower petals 1780, bristles 1782, sports team logos/mascots, fictional characters (e.g., cartoon characters), seasonal decorations (e.g., Christmas tree, snowflake, easter egg, etc.), etc. Further, one embodiment of the cap 170 may include a relatively flat top surface 176 that has a flat decorative element 178 with a plurality of designs, patterns, colors, or textures in any given embodiment of the device 100. In addition, it is also contemplated that the device 100 may be provided in a kit with several removable caps 170 of differing decorative elements 178, which can be placed on the device 100 as the user desires.

FIG. 3 illustrates a perspective view of one potential embodiment of a passenger compartment air vent filtering device 100 of the present invention in accordance with the disclosed architecture, wherein the two filter trays are in the open position so that the filters 130, 150 may be loaded therein. More specifically, in FIG. 3 the placement of the HEPA filter 130 and UV light filter 150 become fully apparent. As referenced above, the body portion 110 has a plurality of covers 1140, wherein each of the covers 1140 enclose one of the HEPA filter 130 and the UV light filter 150 within the body portion 110 of the air filtering device 100. Each cover 1140 conceals an opening 114 that allows each filter 130/150 to be inserted into the hollow body portion 110.

As best shown in FIG. 3, each of the HEPA filter 130 and the UV light filter 150 are arranged within the body portion 110 in a stacked fashion so that air passing through the air filtering device 100 from the bottom 116 must first pass through the HEPA filter 130 and then through the UV light filter 150, or vice versa. This filtering redundancy significantly decreases the possibility that unwanted germs, bacteria, microbes, viruses, diseases and the like will pass through the air filtering device 100 and onto the user from the air vent 10.

In the preferred embodiment of the device 100, the interior of the body portion 110 may have a plurality of tabs or nubs (not shown) so that the HEPA filter 130 and the UV light filter 150 can rest upon them to retain the same in its proper position within the body portion 110. Nonetheless, in an alternative embodiment of the air filtering device 100 it is contemplated that the body portion 110 may include a plurality of repositionable filter trays for holding each of the HEPA filter 130 and the UV light filter 150 so that they can partially extend outwardly from the body portion 100 through each filter opening 114 via a spring loaded sliding track assembly (not shown). Then, each filter 130/150 may be placed into its respective tray, and pushed back into the body portion 110 to retain the filters 130/150 within the body portion 110.

Each of the first and second filters 130, 150 purify the air leaving the air vent 10 prior to entering the passenger compartment. The first filter 130 is preferably a 0.5-micron HEPA filter, which a user may dispose of and replace after several uses. The HEPA filter 130 is generally disc-shaped and comprised of a relatively flat top surface 132, a bottom surface 134 and a side surface 136. The second filter 150 of the air filtering device 100 is preferably a UV light filter 150. The UV light filter 150 is also preferably comprised of a disc-shaped body that has a flat top surface 152 and flat bottom surface 158. However, the filter 150 also includes a continuous opening 154 in the top surface 152 that gives the filter 150 a ring-like shape or body. The inner surface 155 of the filter 150 includes a UV light source 1540 that extends circumferentially around the entire inner surface 155, such that the light source 1540 shines UV light within the body portion 110 to kill airborne pathogens, bacteria, viruses, germs and the like in the air that passes through the body portion 110 and that may have passed through the HEPA filter 110.

The light source 1540 is preferably powered by a battery 160 that may be stored within the UV light filter 150 or elsewhere in the body portion 110. The battery 160 may be a disposable battery or a rechargeable battery, and may be easily replaced or removed if necessary, for recharging. In one embodiment, the batteries can be selected from disposable batteries or rechargeable batteries in sufficient quantity to provide power to the UV light source 1540. Suitable batteries include alkaline, nickel-cadmium, nickel-metal hydride, etc. Throughout this specification the terms “battery”, “battery pack”, and “batteries” may be used interchangeably to refer to one or more wet or dry cells or batteries of cells in which chemical energy is converted into electricity and used as a source of DC power. References to recharging or replacing batteries may refer to recharging or replacing individual cells, individual batteries of cells, or a package of multiple battery cells as deemed appropriate for any given battery technology that may be used. If the battery 160 is rechargeable, it can be recharged by a user via a USB charging port 1560 on an outer surface 156 of the UV light filter 150. In differing embodiments of the air filtering device 100, the port 1560 may be comprised of any number of a plurality of different USB power ports such as, but not limited to, a Mini-USB, Micro-USB, USB-A, USB-C, etc.

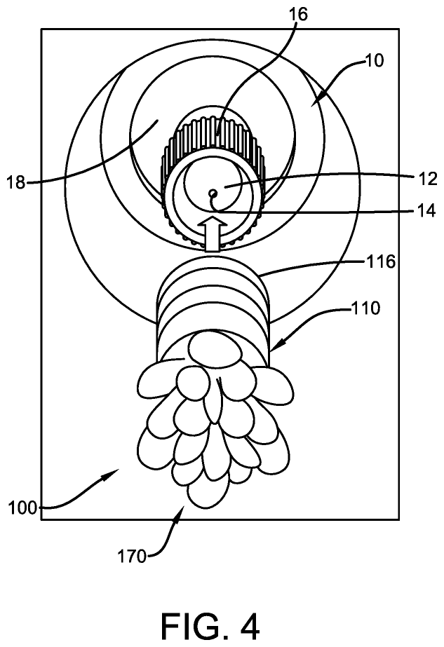

FIG. 4 illustrates a perspective view of one potential embodiment of a passenger compartment air vent filtering device 100 of the present invention in accordance with the disclosed architecture, wherein the filtering device 100 is about to be installed on an air vent 10 in a passenger cabin of a public transportation vehicle. As noted above, the bottom surface 116 of the body portion 110 has a continuous opening therein that is greater in diameter than the diameter of the adjustment ring 16 of the air vent 10 that enables the air filtering device 100 to fit over and attach to the air vent 10. Nonetheless, in an alternative embodiment of the air filtering device 100, a semi-rigid or flexible body portion 110 is provided, and the body portion 110 is able to be stretched around the adjustment ring 16, so that the body portion 110 may have a diameter that is slightly less than that of the outer adjustment ring 16 of the air vent 10. In either embodiment, the body portion 110 forms a seal around the vent 10 such that the vent nipple 12 is completely enclosed and contained within the body portion 110 and any air that exits the vent 10 via the aperture 14 must travel through the body portion 110 and therefore each of the HEPA filter 130 and the UV light filter 150 before exiting the air filtering device 100 via the cap 170 and decorative filter element 178.

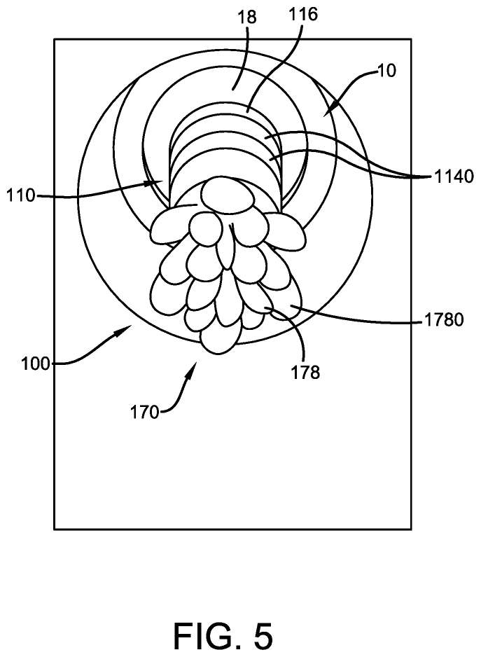

FIG. 5 illustrates a perspective view of one potential embodiment of a passenger compartment air vent filtering device 100 of the present invention in accordance with the disclosed architecture, wherein the filtering device 100 is installed on an air vent 10 in a passenger cabin of a public transportation vehicle. More specifically, once fully secured around the ring 16, the bottom surface 116 makes contact with the spherical body 18 of the vent 10 to secure an airtight connection, as best seen in FIG. 5. Nonetheless, in an alternative embodiment of the air filtering device 100, the bottom surface 116 may have an alternative attachment means, such as a plurality of suction-cups or an adhesive seal, to further secure the bottom surface 116 to the vent body 18 to form an airtight seal.

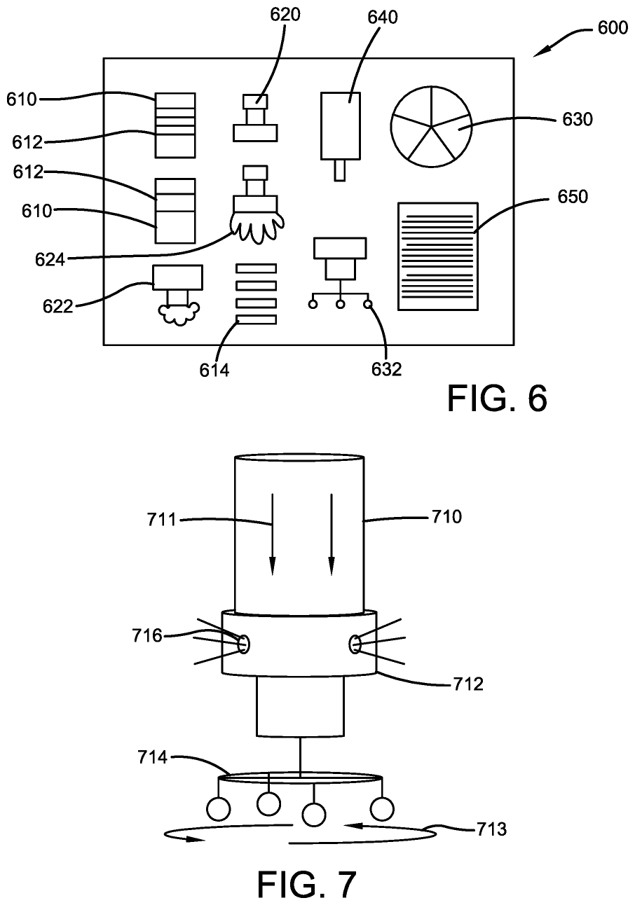

FIG. 6 illustrates a perspective view of one potential embodiment of a passenger compartment air vent filtering device kit of the present invention in accordance with the disclosed architecture. More specifically, FIG. 6 illustrates a retail package 600 that may be acquired at a retail establishment or purchased online. The package 600 has one or more air vent filter covers 610 which include one or more compartments 612 for holding one or more filters 614, such as HEPA filter 130 and/or UV light filter 150. Also included in the package 600 are several examples of the decorative end caps 620, 622, 624, each of which may be placed over the body portion 110 of the air vent filter cover 610. In addition, the package 600 provides alternative end caps 630 and 632 which are a pinwheel and mobile, respectively, and which may move when the air impinges on the end caps 630, 632 to provide entertainment for small children during their commute. The package 600 may also include the battery 160, a scent package 640 for infusing the air passing through the air filtering device with a pleasant scent and instructions 650.

FIG. 7 illustrates a perspective view of one potential embodiment of a passenger compartment air vent filtering device of the present invention in accordance with the disclosed architecture, wherein the cap further comprises an accessory that moves as air is passing therethrough. More specifically, FIG. 7 illustrates a decorative and movable element 712 disposed on the end of the air vent 710, such as a mobile. When the air flow 711 passing through the air filtering device impinges on the decorative element 712, the air flow 711 causing the decorative element to move in a generally circular direction 713. FIG. 7 also illustrates a number of LED lights 716 which may be used to provide illumination to the user in any number of different ways, and which may be in electrical communication with, and powered by, the battery 160 or which may contain their own battery (not shown).

Notwithstanding the forgoing, the passenger compartment air vent filtering device 100 can by any suitable size, shape, and configuration as is known in the art without affecting the overall concept of the invention, provided that it accomplishes the above stated objectives. One of ordinary skill in the art will appreciate that the shape and size of the air vent filtering device 100 and its various components, as show in the FIGS. are for illustrative purposes only, and that many other shapes and sizes of the air vent filtering device 100 are well within the scope of the present disclosure. Although dimensions of the air vent filtering device 100 and its components (i.e., length, width, and height) are important design parameters for good performance, the air vent filtering device 100 and its various components may be of any shape or size that ensures optimal performance during use and/or that suits user need and/or preference.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A passenger compartment air vent cover comprising:
a body portion sized and configured to fit over an air vent in a passenger compartment, wherein the body portion comprises a first end, a second end and an internal passageway extending between the first end and the second end to allow a flow of air to pass from the air vent through the internal passageway;
a first filter positioned in the internal passageway;
a second filter positioned in the internal passageway; and
an end cap sized and configured to fit over the second end of the body portion, wherein the flow of air passes through the end cap.

2. The passenger compartment air vent cover as recited in claim 1, wherein the first filter is a HEPA filter.

3. The passenger compartment air vent cover as recited in claim 2, wherein the second filter is a UV light filter.

4. The passenger compartment air vent cover as recited in claim 3 further comprising a battery for supplying power to the UV light filer.

5. The passenger compartment air vent cover as recited in claim 4, wherein at least one of the first filter, the second filter and the end cap are infused with a scent.

6. The passenger compartment air vent cover as recited in claim 5, wherein the end cap comprises a repositionable element that articulates when it comes into contact with the flow of air.

7. The passenger compartment air vent cover as recited in claim 5, wherein the scent is selected from a group including a lavender, a peppermint, a spearmint, a citrus, an eucalyptus, a clove, a tea tree, an evergreen, a sandalwood, a vanilla, a cedarwood or a combination thereof.

8. The passenger compartment air vent cover as recited in claim 1, wherein the body portion is comprised of a flexible material.

9. The passenger compartment air vent cover as recited in claim 2, wherein the HEPA filter is a 0.5-micron HEPA filter.

10. The passenger compartment air vent cover as recited in claim 1 further comprising a battery and at least one LED light.

11. An air filter cover for an air vent opening in a passenger compartment, the air filter cover comprising:
a body portion having a first end, a second end and a continuous opening extending between the first end and the second end, wherein the first end is sized to fit over the air vent opening;
at least one filter positioned within the continuous opening; and
a decorative element disposed over the second end of the body portion, wherein the decorative element is selected from a group including a flower, a plurality of bristles, a character, a geometric shape, an accessory, a moveable element, and a combination thereof.

12. The air filter cover as recited in claim 11 comprising a first filter and a second filter, wherein the first filter is a HEPA filter that is positioned in the continuous opening between the first end and the second filter.

13. The air filter cover as recited in claim 12 further comprising a battery, wherein the second filter is a UV light filter that is powered by the battery.

14. The air filter cover as recited in claim 13, wherein the HEPA filter is a 0.5-micron HEPA filter.

15. The air filter cover as recited in claim 11, wherein the moveable element is a pinwheel or a mobile.

16. The air filter cover as recited in claim 13, wherein the accessory is a plurality of LED lights that are powered by the battery.

17. An air filtering kit for use in a passenger compartment, the air filtering kit comprising:
a package for holding a number of components for use with an air vent in the passenger compartment, wherein the number of components are comprised of:
a body portion having a first end, a second end and a continuous opening extending from the first end to the second end;
a plurality of HEPA filters;
a plurality of UV light filters;
a battery;
a scented solution; and
a plurality of decorative end caps for removable attachment to the second end of the body portion.

18. The air filtering kit for use in a passenger compartment as recited in claim 17, wherein at least one of the plurality of HEPA filters and at least one of the plurality of UV light filters are positioned in the continuous opening and filter a flow of air passing therethrough.

19. The air filtering kit for use in a passenger compartment as recited in claim 17 further comprising a plurality of LED lights that are powered by the battery and a USB port.

20. The air filtering kit for use in a passenger compartment as recited in claim 17, wherein the battery is in electrical communication with at least one of the plurality of UV light filters.
